(19) United States
(12) Patent Application Publication
(10) Pub. No.: US 2026/0191504 A1
(43) Pub. Date: Jul. 9, 2026

(54) SINGLE MOTOR DATA COLLECTION SCANNERS

(71) Applicant: Verathon Inc., Bothell, WA (US)

(72) Inventors: Quinn Dickenson, Bellevue, WA (US); Christian Fabian Martinez, Mill Creek, WA (US)

(73) Assignee: Verathon Inc., Bothell, WA (US)

(21) Appl. No.: 19/442,131

(22) Filed: Jan. 7, 2026

Related U.S. Application Data
(60) Provisional application No. 63/742,953, filed on Jan. 8, 2025.

(51) Int. Cl. A61B 8/00

(52) U.S. Cl. CPC A61B 8/4461

(57) ABSTRACT

In some embodiments, a system may include a motor having a shaft. The system may also include a body having a first gear having a first plurality of teeth. The system may also include a stand. The system may also include an assembly rotatably coupled to the stand. The assembly may comprise a second gear having a second plurality of teeth. The system may also include a third gear having a third plurality of teeth. The third gear may be coupled to the shaft. The third plurality of teeth may mesh with the second plurality of teeth. The system may also include a fourth gear having a fourth plurality of teeth. The fourth plurality of teeth may mesh with the first plurality of teeth. The system may also include a friction member biased between the stand and the fourth gear.

### CROSS-REFERENCE TO RELATED APPLICATIONS

This application claims benefit under 35 U.S.C. § 119 (e) to U.S. Provisional Application No. 63/742,953, filed Jan. 8, 2025, entitled “Single Motor Data Collection Scanners,” the disclosure of which is incorporated herein by reference in its entirety.

### FIELD OF DISCLOSURE

The disclosed systems and methods are related to the field of medical devices used in medical procedures and diagnostics. More particularly, the disclosed systems and methods are concerned with data collection scanners of the medical devices.

### BACKGROUND

Medical devices, such as bladder scanners or other volume determination sensors are configured to scan a portion of a patient's anatomy. Rotation of the sensor in multiple degrees of freedom is required to accurately determine the volume of a specific portion of a patient's anatomy. Conventionally, scanners require independent motors for each degree of freedom the scanner is configured to rotate in. For example, a scanner configured to determine the volume of a specific portion of a patient's anatomy typically requires a first motor configured to rotate the sensor in a first degree of freedom and a second motor configured to rotate the sensor in a second degree of freedom. However, scanners having independent motors for each degree of freedom are large, heavy, and expensive.

### SUMMARY

In some embodiments, a system may include a motor having a shaft. The system may also include a body comprising a first gear having a first plurality of teeth. The system may also include a stand. The system may also include an assembly rotatably coupled to the stand. The assembly may comprise a second gear having a second plurality of teeth. The system may also include a third gear having a third plurality of teeth. The third gear may be coupled to the shaft. The third plurality of teeth may mesh with the second plurality of teeth. The system may also include a fourth gear having a fourth plurality of teeth. The fourth plurality of teeth may mesh with the first plurality of teeth. The system may also include a friction member biased between the stand and the fourth gear.

In some embodiments, a scanner system may include a motor having a shaft. The scanner system may also include a body having a first gear having a first plurality of teeth. The scanner system may also include a stand. The scanner system may also include an assembly rotatably coupled to the stand. The assembly may include a sensor and a second gear having a second plurality of teeth. The scanner system may also include a third gear having a third plurality of teeth. The third gear may be coupled to the shaft. The third plurality of teeth may mesh with the second plurality of teeth. The scanner system may also include a fourth gear having a fourth plurality of teeth. The fourth plurality of teeth may mesh with the first plurality of teeth. The scanner system may also include a friction member biased between the stand and the fourth gear. The motor may be a stepper motor and may be configured to apply a plurality of torque values to the third gear via the shaft.

In some embodiments, a method of performing a plurality of scans in a three-dimensional space may include operating a motor at a first torque value in a first rotational direction to thereby rotate a sensor about a first axis in a first o rotational direction. The sensor may perform a predetermined number N of scans during the rotation about the first axis in the first o rotational direction. The method may also include operating the motor at a second torque value in the first rotational direction to thereby rotate the sensor about a second axis in a first θ rotational direction. The method may also include operating the motor at the first torque value in a second rotational direction to thereby rotate the sensor about the first axis in a second φ rotational direction. The sensor may perform a predetermined number M of scans during the rotation about the first axis in the second φ rotational direction. The method may also include operating the motor at the second torque value in the second rotational direction to thereby rotate the sensor about the second axis in a second θ rotational direction.

A method of performing a plurality of scans in a three-dimensional space may use a single motor scanner system having a motor shaft, a body comprising a first gear having a first plurality of teeth, a stand, and an assembly rotatably coupled to the stand. The assembly may have a sensor and a second gear having a second plurality of teeth. The scanner system may also include a third gear having a third plurality of teeth. The third gear may be coupled to the motor shaft and the third plurality of teeth may mesh with the second plurality of teeth. The scanner system may also include a fourth gear having a fourth plurality of teeth. The fourth plurality of teeth may mesh with the first plurality of teeth. The scanner system may also include a friction member biased between the stand and the fourth gear.

The method may include the steps of operating the motor at a first torque value in a first rotational direction to thereby rotate the sensor about a first axis in a first φ rotational direction. The sensor may perform a predetermined number N of scans during the rotation about the first axis in the first φ rotational direction. The method may also include operating the motor at a second torque value in the first rotational direction to thereby rotate the sensor about a second axis in a first θ rotational direction. The method may also include operating the motor at the first torque value in a second rotational direction to thereby rotate the sensor about the first axis in a second φ rotational direction. The sensor may perform a predetermined number M of scans during the rotation about the first axis in the second φ rotational direction. The method may also include operating the motor at the second torque value in the second rotational direction to thereby rotate the sensor about the second axis in a second θ rotational direction.

### BRIEF DESCRIPTION OF THE DRAWINGS

The features and advantages of the present disclosure will be more fully disclosed in, or rendered obvious by, the following detailed descriptions of example embodiments. The detailed descriptions of the example embodiments are to be considered together with the accompanying drawings wherein like numbers refer to like parts and further wherein:

FIG. 1 illustrates an isometric view of a scanner system in accordance with some embodiments;

FIG. 2 illustrates a motor of a scanner system in accordance with some embodiments;

FIG. 3 illustrates a body of a scanner system in accordance with some embodiments;

FIG. 4 illustrates a stand of a scanner system in accordance with some embodiments;

FIG. 5 illustrates a pinion gear of a scanner system in accordance with some embodiments;

FIG. 6 illustrates a planetary gear of a scanner system in accordance with some embodiments;

FIG. 7A illustrates a first example of an assembly of a scanner system in accordance with some embodiments;

FIG. 7B illustrates a second example of an assembly of a scanner system in accordance with some embodiments;

FIG. 8 illustrates a sensor of a scanner system in accordance with some embodiments;

FIG. 9 illustrates an exploded view of a scanner system in accordance with some embodiments;

FIG. 10 illustrates a cross-sectional view of a scanner system in accordance with some embodiments;

FIG. 11 illustrates a block diagram of an exemplary computing device of a scanner system in accordance with some embodiments;

FIG. 12 illustrates an isometric view of a scanner system in accordance with some embodiments;

FIG. 13 illustrates a side view of a scanner system in accordance with some embodiments;

FIG. 14 illustrates an exemplary predetermined sequence of a scanner system in accordance with some embodiments;

FIG. 15 illustrates a block diagram of a first exemplary method of performing a plurality of scans in a three-dimensional space in accordance with some embodiments; and

FIG. 16 illustrates a block diagram of a second exemplary method of performing a plurality of scans in a three-dimensional space in accordance with some embodiments.

While the present disclosure is susceptible to various modifications and alternative forms, specific embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the disclosure as defined by the appended claims.

### DETAILED DESCRIPTION

This description of the exemplary embodiments is intended to be read in connection with the accompanying drawings, which are to be considered part of the entire written description. It should be understood, however, that the present disclosure is not intended to be limited to the particular forms disclosed and that the drawings are not necessarily shown to scale. Rather, the present disclosure covers all modifications, equivalents, and alternatives that fall within the spirit and scope of these exemplary embodiments. In the description, relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The terms “couple,” “coupled,” “operatively coupled,” “operatively connected,” and the like should be broadly understood to refer to connecting devices or components together either mechanically, or otherwise, such that the connection allows the pertinent devices or components to operate with each other as intended by virtue of that relationship.

The present disclosure includes various embodiments of a scanner system that may be configured to scan a portion of a patient's anatomy. Exemplary aspects of the scanner system include a single motor configured to rotate in a plurality of degrees of freedom. The scanner systems disclosed herein overcome the disadvantages of conventional scanner systems and allow for a smaller, lighter, and cheaper scanner system over conventional alternatives.

The scanner systems disclosed herein may be used and adapted for a variety of different medical devices, procedures, and diagnostics. For example, the scanner systems disclosed herein may be used to scan a patient's heart, blood vessels, eyes, thyroid, brain, breast, abdominal organs (e.g., liver, gall bladder, spleen, stomach, colon, small intestine, appendix, and bladder), skin, reproductive organs, and muscles just to provide a few non-limiting examples. The scanner systems may also be scaled based on the patient's characteristics. For example, the scanner systems may be configured based on the type of patient (e.g., human or animal), the patient's age (e.g., pediatric or adult), the patient's size (e.g., small, medium, large, etc.), and/or a specific portion of a patient's anatomy as discussed above, just to provide a few non-limiting examples.

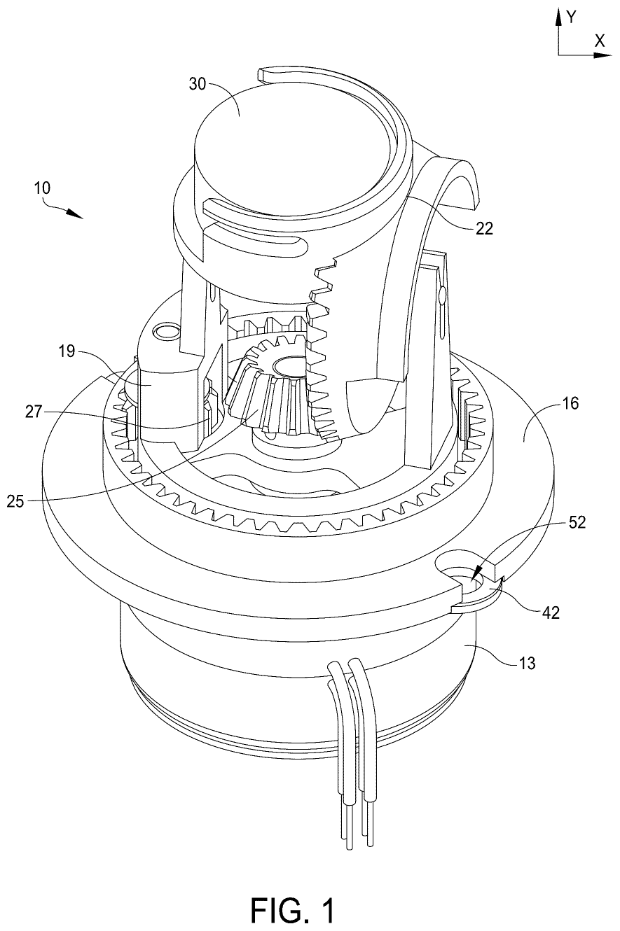

Referring now to the figures, FIG. 1 illustrates an isometric view of a scanner system 10 in accordance with some embodiments. Scanner system 10 may include a motor 13, a body 16, a stand 19, an assembly 22, a pinion gear 25, a planetary gear 27, and a sensor 30. According to some embodiments, sensor 30 is configured to scan a portion of a patient's anatomy in a plurality of degrees of freedom, facilitated by the motor 13, the body 16, the stand 19, the assembly 22, the pinion gear 25, and the planetary gear 27 as disclosed herein. In some embodiments, the scanner system 10 includes a cap or housing to contain the scanner system 10 and allow for the moving parts of the scanner system 10 to be immersed in oil or other suitable lubricant.

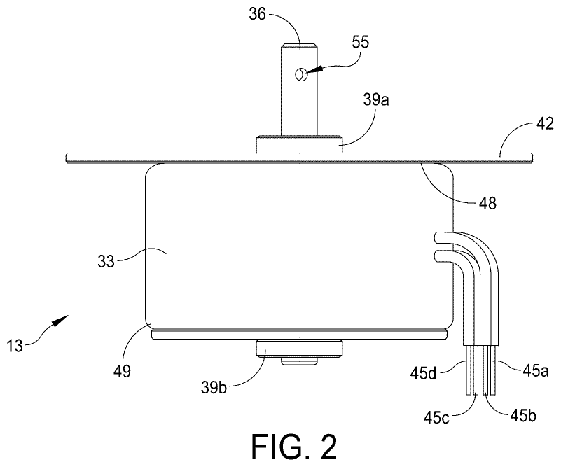

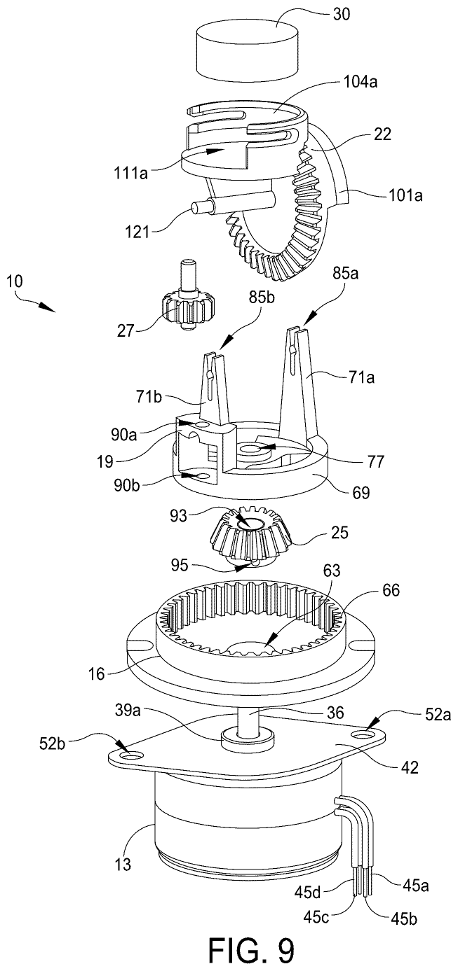

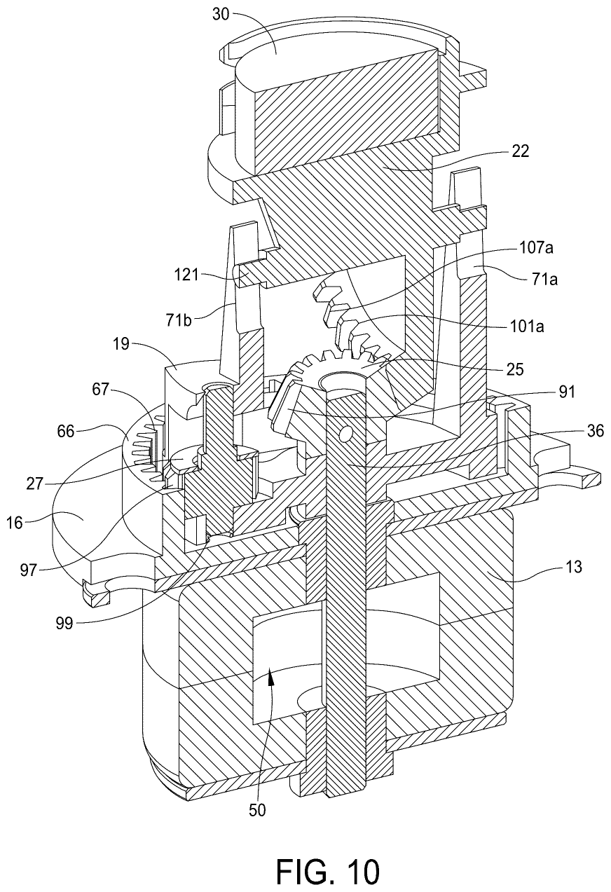

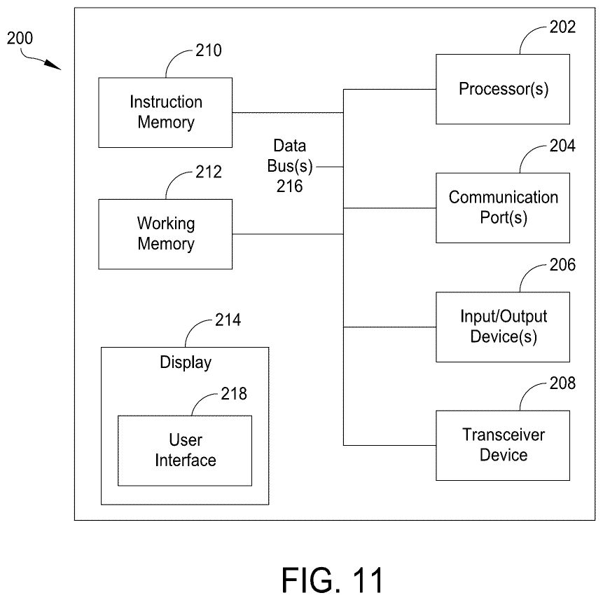

FIG. 2 illustrates the motor 13 of the scanner system 10 in accordance with some embodiments. The motor 13 may include a housing 33, a shaft 36, one or more bearings 39a-b, a mount 42, and one or more wires 45a-d. The housing 33 may extend between a first end 48 and a second end 49. The housing 33 may define a void 50 extending between the first end 48 and the second end 49 that is sized and configured to receive the shaft 36 therein, as best seen in FIG. 10. The bearing 39a may be coupled to the shaft 36 at the first end 48 of the housing 33 and the bearing 39b may be coupled to the shaft 36 at the second end 49 of the housing 33. The one or more bearings 39a-b may be configured to facilitate smooth rotation of the shaft 36 while the motor 13 is operating. The mount 42 may be coupled to the first end 48 of the housing 33 and the body 16. In some embodiments, the mount 42 may define one or more slots 52a-b, as best seen in FIGS. 1 and 9, that is sized and configured to receive a fixation element (e.g., bolt, nail, screw, pin, etc.) such that scanner system 10 may be fixedly coupled to a surface, such as the inside of a medical device.

The one or more wires 45a-d may provide suitable power to operate motor 13. For example, the one or more wires 45a-d provide sufficient alternating current (AC) or direct current (DC) power to the motor 13 to rotate the shaft 36. The shaft 36 may include a hole 55 sized and configured to receive a fastener (e.g., bolt, nail, screw, pin, etc.) used to couple the pinion gear 25 to the shaft 36.

The motor 13 may be any suitable motor to rotate one or more gears. For example, motor 13 may be a DC motor, an AC motor, or other special purpose motor such as a stepper motor, brushless motor, hysteresis motor, reluctance motor, or universal motor just to provide a few non-limiting examples. In some embodiments, the motor 13 is controlled with a current driven controller or a voltage driven controller. In some embodiments, the motor 13 may be configured to rotate the shaft 36 at 100-3600 revolutions per minute (RPM). It will be appreciated that the shaft RPM may be less than 100 or more than 3600 in some embodiments. The motor 13 may be configured to apply a plurality of torque values to the shaft 36 according to a predetermined sequence. For example, the motor 13 may be configured to apply a first torque value in a first mode of operation and a second torque value in a second mode of operation. As an example, the first torque value may be 5 Newton-millimeters (N·mm) and the second torque value may be 7 N·mm. It will be appreciated that the first torque value and the second torque value may be less than 5 N·mm or more than 7 N·mm in some embodiments.

In some embodiments, the motor 13 may be operated with one or more buttons on the medical device the scanner system 10 supports, remotely operated from a controller separate from the medical device, or by a robotic system communicatively coupled to a computing device as will be discussed in further detail below.

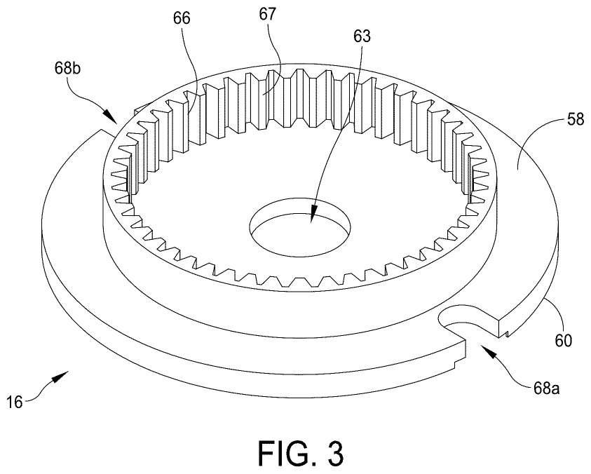

FIG. 3 illustrates the body 16 of the scanner system 10 in accordance with some embodiments. The body 16 may extend between a top portion 58 and a bottom portion 60. The body 16 may define an aperture 63 that is sized and configured to receive the shaft 36 therein. The top portion 58 includes a ring gear 66 that defines a plurality of teeth 67 configured to mesh with teeth from another gear. The ring gear 66 is sized such that the stand 19 can be disposed within the ring gear 66. In some embodiments, the body 16 defines one or more slots 68a-b that are configured to align with the one or more slots 52a-b on the mount 42, as illustrated in FIG. 1, so that the scanner system 10 can be secured to a surface with a respective fastener.

In some embodiments, the body 16 may be fixedly coupled to the mount 42, such as welded to it or formed integrally with it for example. In other embodiments, the body 16 is removably coupled to the mount 42, such as with an adhesive, a press-fit connection, or a fastener. In some embodiments, the body 16 may be generally circular in shape as illustrated in FIG. 3. The ring gear 66 may also be generally circular in shape so as to generally match the shape of the body 16. However, it will be appreciated that the body 16 and/or the ring gear 66 may be any other suitable shape (e.g., square, rectangular, triangular, or some other polygonal shape). In some embodiments, the body 16 and the ring gear 66 are different shapes.

The body 16 may be made of any suitable material, such as metal, metal alloy, plastic, nylon, etc. In some embodiments, the body 16 may be made of mixed materials. For example, the ring gear 66 may be made of a first material and the rest of the body 16 extending between the top portion 58 and the bottom portion 60 is made of a second material. In some embodiments, the ring gear 66 is made of a lubricous plastic to facilitate smooth gear rotation and engagement. In some embodiments, the body 16 may be injection molded or 3D printed. One of ordinary skill in the art will appreciate other materials suitable for the body 16 and ring gear 66, and the materials provided above are merely non-limiting examples.

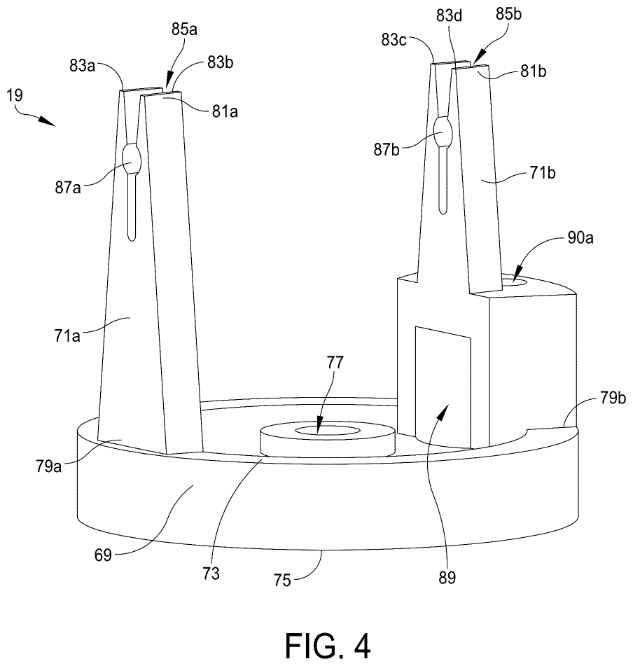

FIG. 4 illustrates the stand 19 of the scanner system 10 in accordance with some embodiments. The stand 19 may include a support 69 and one or more posts 71a-b. The support 69 may extend between a top portion 73 and a bottom portion 75. The one or more posts 71a-b may be coupled to the support 69 and extend away from the top portion 73 of the support 69. The stand 19 may also define an aperture 77 that is sized and configured to receive the shaft 36 therein. The stand 19 may be configured to be placed within the ring gear 66. For example, the support 69 may be shaped similar to the ring gear 66 such that the support 69 fits within the ring gear 66 as best seen in FIG. 1.

The first post 71a may extends between a first end 79a and a second end 81a. The second end 81a of the first post 71a may define a pair of ledges 83a-b. The pair of ledges 83a-b may be spaced apart, defining a void 85a. In some embodiments, the void 85a extends down to a shoulder 87a. The second post 71b extends between a first end 79b and a second end 81b. The second end 81b of the second post 71b may define a pair of ledges 83c-d. The pair of ledges 83c-d may be spaced apart, defining a void 85b. In some embodiments, the void 85b extends down to a shoulder 87b. The second post 71b may also define a space 89 near the first end 79b. The space 89 may be sized and configured to receive the planetary gear 27 therein. The second post 71b may also define one or more openings 90a-b configured to receive a portion of the planetary gear 27 to secure the planetary gear 27 within the space 89, as best seen in FIG. 9.

The stand 19 may be made of any of any suitable material, such as metal, metal alloy, plastic, nylon, etc. In some embodiments, the stand 19 may be injection molded or 3D printed. One of ordinary skill in the art will appreciate other materials suitable for the stand 19 and the materials provided above are merely non-limiting examples.

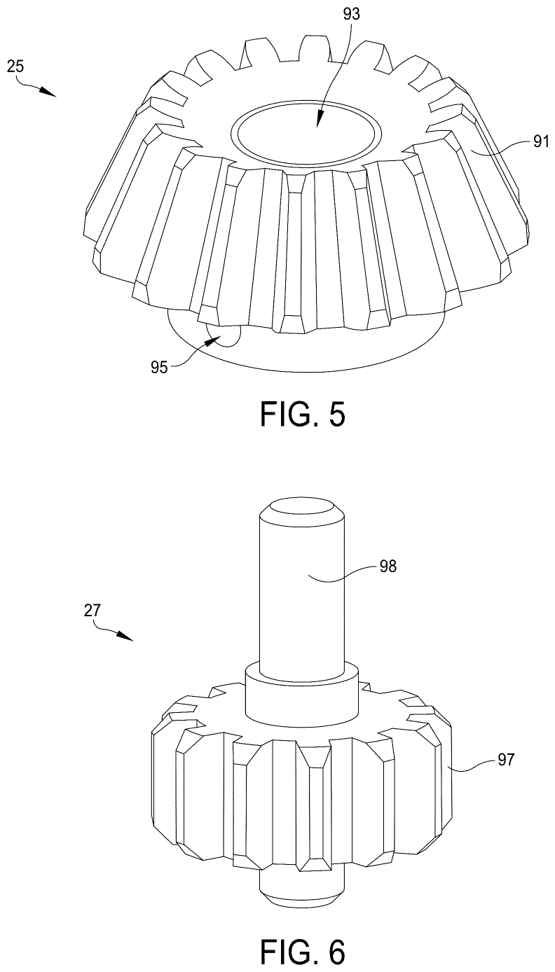

FIG. 5 illustrates the pinion gear 25 of the scanner system 10 in accordance with some embodiments. The pinion gear 25 may define a plurality of teeth 91 configured to mesh with teeth from another gear. The pinion gear 25 may also define an aperture 93 that is sized and configured to receive the shaft 36. The pinion gear 25 may also define a hole 95 that is sized and configured to receive a fastener (e.g., bolt, nail, screw, pin, etc.) used to couple the pinion gear 25 to the shaft 36. For example, the fastener may be disposed through both the hole 55 and hole 95, coupling the pinion gear 25 to the shaft 36.

The pinion gear 25 may be made of any suitable material, such as metal, metal alloy, plastic, nylon, etc. In some embodiments, the pinion gear 25 may be made of a lubricous plastic to facilitate smooth gear rotation and engagement. In some embodiments, the pinion gear 25 may be injection molded or 3D printed. One of ordinary skill in the art will appreciate other materials suitable for the pinion gear 25 and the materials provided above are merely non-limiting examples.

FIG. 6 illustrates the planetary gear 27 of the scanner system 10 in accordance with some embodiments. The planetary gear 27 may define a plurality of teeth 97 configured to mesh with the plurality of teeth 67 of the ring gear 66. The planetary gear 27 may also include a pin 98. The pin 98 may be sized and configured to be disposed within the one or more openings 90a-b of the second post 71b to secure the planetary gear 27 within the space 89. As best seen in FIG. 10, the planetary gear 27 may be biased against a friction member 99 between the pin 98 and the support 69 in some embodiments. In some embodiments, the friction member 99 may be a spring or a ratchet and pawl mechanism configured to secure the planetary gear 27 in certain modes of operation.

The planetary gear 27 may be made of any suitable material, such as metal, metal alloy, plastic, nylon, etc. In some embodiments, the planetary gear 27 may be made of a lubricous plastic to facilitate smooth gear rotation and engagement. In some embodiments, the planetary gear 27 may be injection molded or 3D printed. One of ordinary skill in the art will appreciate other materials suitable for the planetary gear 27 and the materials provided above are merely non-limiting examples.

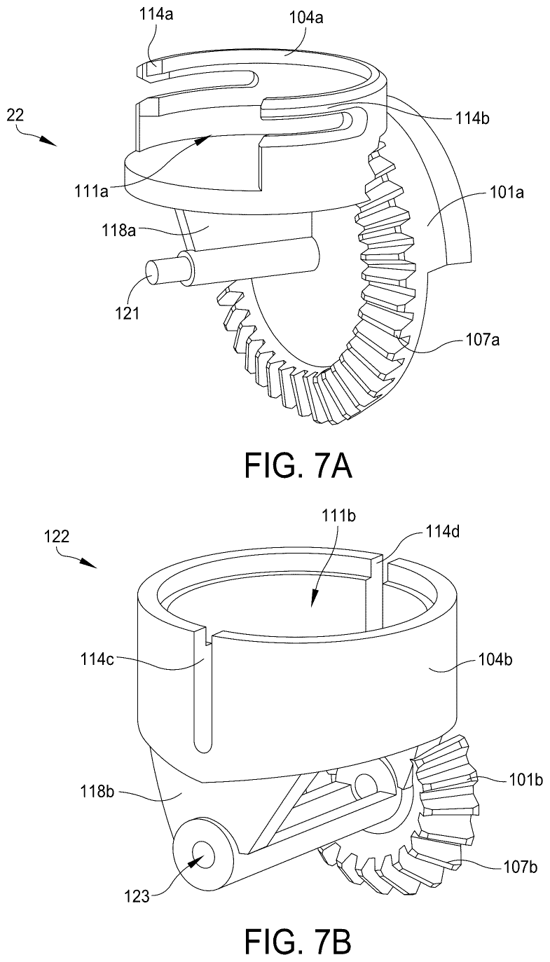

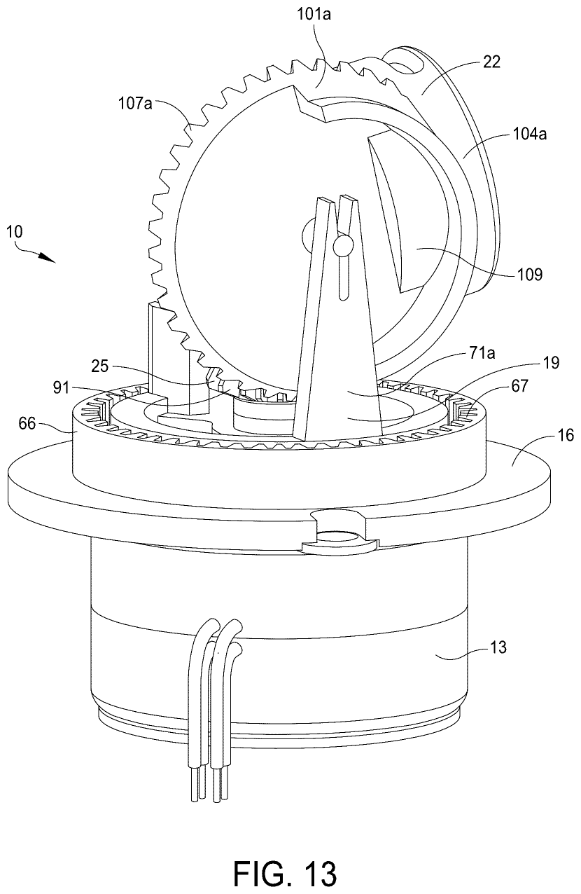

FIG. 7A illustrates a first example of an assembly 22 of the scanner system 10 in accordance with some embodiments. The assembly 22 may include a bevel gear 101a and a basket 104a. The bevel gear 101a may define a plurality of teeth 107a configured to mesh with the plurality of teeth 91 of the pinion gear 25. The bevel gear 101a may also define a stop 109, as best seen in FIG. 13. The stop 109 is configured to stop rotation of the bevel gear 101a at a predetermined angle.

The basket 104a may define a space 111a that is sized and configured to receive the sensor 30. In some embodiments, the basket 104a may define one or more ridges 114a-b configured to secure the sensor 30 within the space 111a and facilitate easy removal or replacement of the sensor 30. As best illustrated in FIG. 7A, the space 111a may be partially open such that the sensor 30 can be slid into the basket 104a from the side.

The basket 104a may include a swivel joint 118a having a rod 121 disposed through the bevel gear 101a to pivotably couple the assembly 22 to the stand 19. The rod 121 may be sized and configured to be disposed within the voids 85a-b of the posts 71a-b. For example, a portion of the rod 121 may be configured to sit on the shoulders 87a-b disposed within the voids 85a-b. The basket 104a may also be coupled to the bevel gear 101a, such that movement of the bevel gear 101a facilitates movement, or rotation, of the basket 104a about a first axis.

FIG. 7B illustrates a second example of an assembly 122 of the scanner system 10 in accordance with some embodiments. The assembly 122 may be similar to the assembly 22 as discussed above. The assembly 122 may include a bevel gear 101b and a basket 104b. The bevel gear 101b may define a plurality of teeth 107b configured to mesh with the plurality of teeth 91 of the pinion gear 25. The assembly 122 may also define a stop, which is configured to restrict motion of the bevel gear 101b. For example, the stop may use a hard stop, such as stop 109 as best illustrated in FIG. 13, or may use a force applied to restrict motion of the bevel gear 101b in the phi direction, such as with a spring, through friction, some other biasing member, etc.

The basket 104b may define a space 111b sized and configured to receive the sensor 30. In some embodiments, the basket 104b may define one or more ridges 114c-d configured to secure the sensor 30 within the space 111b and facilitate easy removal or replacement of the sensor 30. As best illustrated in FIG. 7B, the space 111b may be open at one end such that the sensor 30 can be placed into the basket 104b from above.

The basket 104b may include a swivel joint 118b, which may define a hole 123 configured to receive a rod (e.g., rod 121) to secure the assembly 122 to the stand 19. The rod may be sized and configured to be disposed within the voids 85a-b of the posts 71a-b. For example, a portion of the rod may be configured to sit on the shoulders 87a-b disposed within the voids 85a-b. The basket 104b may also be coupled to the bevel gear 101b, such that movement of the bevel gear 101b facilitates movement, or rotation, of the basket 104b in a first axis.

The assemblies 22, 122 be made of any suitable material, such as metal, metal alloy, plastic, nylon, etc. In some embodiments, the assemblies 22, 122 may be made of mixed materials. For example, the bevel gears 101a-b may be made of a first material and the baskets 104a-b may be made of a second material. In some embodiments, the bevel gears 101a-b are made of a lubricous plastic to facilitate smooth gear rotation and engagement. In some embodiments, the bevel gears 101a-b and/or the baskets 104a-b may be injection molded or 3D printed. One of ordinary skill in the art will appreciate other materials suitable for the bevel gears 101a-b and the baskets 104a-b, and the materials provided above are merely non-limiting examples.

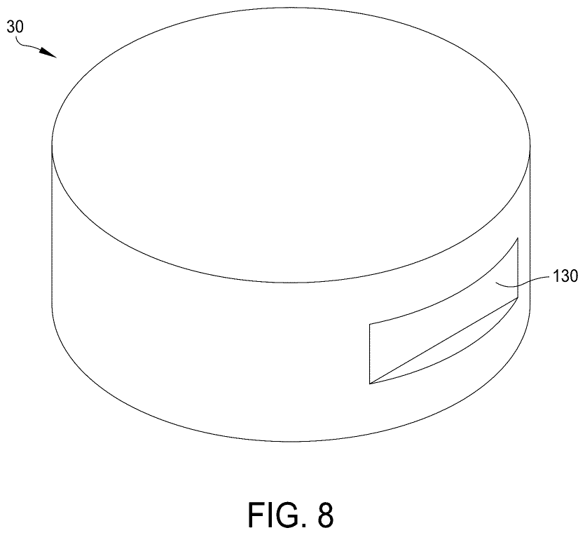

FIG. 8 illustrates the sensor 30 of the scanner system 10 in accordance with some embodiments. In some embodiments, the sensor 30 may define one or more grooves 130 that are sized and configured to engage the ridges 114a-b or ridges 114c-d. The sensor 30 may be any suitable sensor, such as an ultrasound probe or other suitable sensor for medical applications. In some embodiments, the sensor 30 may be an optical sensor or a coder sensor. In some embodiments, the sensor 30 is an ultrasound probe with an operating frequency of 2.5-3.5 MHz, 2-4 MHz, or 1-5 MHz. In some embodiments, the sensor 30 is an ultrasound probe with an operating frequency of about 2.9 MHz. It will be appreciated that the sensor 30 may have an operating frequency of less than 1 MHz or greater than 5 MHz in some embodiments.

FIG. 9 illustrates an exploded view of the scanner system 10 in accordance with some embodiments. As discussed above, the scanner system 10 may be associated with or disposed within a medical device, such as a bladder scanner. When assembled, the motor 13 maybe secured within the medical device, such as with fasteners through the one or more slots 52a-b of the mount 42. The aperture 63 of the body 16 may be sized and configured to receive the shaft 36. The bottom portion 60 of the body 16 may be configured to sit on the bearing 39a.

The aperture 77 of the stand 19 may be configured to receive the shaft 36, such that the stand 19 is disposed within the ring gear 66. The aperture 93 of the pinion gear 25 may also be configured to receive the shaft 36. A fastener may then be placed through the hole 55 of the shaft 36 and hole 95 of the pinion gear 25, coupling the pinion gear 25 to the shaft 36 and securing the stand 19 within the ring gear 66. The one or more openings 90a-b of the second post 71b may be configured to receive the pin 98 of the planetary gear 27, securing the planetary gear 27 within the space 89.

The voids 85a-b may be configured to receive the rod 121 of the assembly 22, such that the assembly 22 is pivotably coupled to the stand 19, but still free to rotate in a second axis. The space 111a of the basket 104a may be configured to receive the sensor 30, such that movement of the stand 19 rotates the sensor 30 about the first axis and movement of the bevel gear 101a rotates the sensor 30 about the second axis.

FIG. 10 illustrates a cross-sectional view of the scanner system 10 in accordance with some embodiments. When assembled, the plurality of teeth 91 of the pinion gear 25 may be meshed with the plurality of teeth 107a of the bevel gear 101a. Rotation of the shaft 36 by the motor 13 in a first mode of operation (or first torque value) rotates the pinion gear 25 and the bevel gear 101a through the meshing of the plurality of teeth 91 and 107a, such that sensor 30 rotates about the second axis until the stop 109 engages the second post 71b. The plurality of teeth 67 of the ring gear 66 may be meshed with the plurality of teeth 97 of the planetary gear 27. Rotation of the shaft 36 by the motor 13 in a second mode of operation (or at a second torque value) applies torque to the stand 19 so that the fiction force of the friction member 99 is overcome. With the friction force overcome, the planetary gear 27 may move through the meshing of the plurality of teeth 91 and 67, moving the stand 19 within the ring gear 66 so that the sensor 30 rotates about the first axis.

FIG. 11 illustrates a block diagram of an exemplary computing device 200 of a scanner system 10 in accordance with some embodiments. The computing device 200 may be employed by a disclosed system or used to execute a disclosed method. The computing device 200, such as a computing device associated with a medical device, can implement, for example, one or more of the functions described herein. It should be understood, however, that other computing device configurations are possible.

The computing device 200 may include one or more processors 202, one or more communication port(s) 204, one or more input/output devices 206, a transceiver 208, instruction memory 210, working memory 212, and optionally a display 214, all operatively coupled to one or more data buses 216. The data buses 216 may allow for communication among the various devices, the processor(s) 202, the instruction memory 210, the working memory 212, the communication port(s) 204, and/or the display 214. The data buses 216 may include wired, or wireless, communication channels. The data buses 216 may be connected to one or more devices.

The processor(s) 202 may include one or more distinct processors, each having one or more cores. Each of the distinct processors can have the same or different structures. The processor(s) 202 may include one or more central processing units (CPUs), one or more graphics processing units (GPUs), application specific integrated circuits (ASICs), digital signal processors (DSPs), and the like.

The processor(s) 202 may be configured to perform a certain function or operation by executing code, stored on the instruction memory 210, embodying the function or operation of the scanner system 10 and discussed below. For example, the processor(s) 202 may be configured to perform one or more of any function, method, or operation disclosed herein.

The communication port(s) 204 may include, for example, a serial port such as a universal asynchronous receiver/transmitter (UART) connection, a Universal Serial Bus (USB) connection, or any other suitable communication port or connection. In some examples, the communication port(s) 204 may allow for the programming of executable instructions in the instruction memory 210. In some examples, the communication port(s) 204 may allow for the transfer, such as uploading or downloading, of data.

The input/output devices 206 may include any suitable device that allows for data input or output. For example, the input/output devices 206 may include one or more of a keyboard, a touchpad, a mouse, a stylus, a touchscreen, a physical button, a speaker, a microphone, or any other suitable input or output device.

The transceiver 208 may allow for communication with a network, such as a Wi-Fi network, an Ethernet network, a cellular network, or any other suitable communication network. For example, if operating in a cellular network, the transceiver 208 may be configured to allow communications with the cellular network. The processor(s) 202 may be operable to receive data from, or send data to, a network via the transceiver 208.

The instruction memory 210 may include an instruction memory 210 that can store instructions that can be accessed (e.g., read) and executed by the processor(s) 202. For example, the instruction memory 210 may be a non-transitory, computer-readable storage medium such as a read-only memory (ROM), an electrically erasable programmable read-only memory (EEPROM), flash memory, a removable disk, CD-ROM, any non-volatile memory, or any other suitable memory with instructions stored thereon. For example, the instruction memory 210 may store instructions that, when executed by the one or more processors 202, cause the one or more processors 202 to perform one or more of the operations of the scanner system 10.

In addition to the instruction memory 210, the computing device 200 may also include a working memory 212. The processor(s) 202 may store data to, and read data from, the working memory 212. For example, the processor(s) 202 may store a working set of instructions to the working memory 212, such as instructions loaded from the instruction memory 210. The processor(s) 202 may also use the working memory 212 to store dynamic data created during the operation of the computing device 200. The working memory 212 may be a random access memory (RAM) such as a static random access memory (SRAM) or dynamic random access memory (DRAM), or any other suitable memory.

The display 214 may be configured to display user interface 218. The user interface 218 may enable user interaction with the computing device 200. In some examples, a user may interact with the user interface 218 by engaging the input/output devices 206. In some examples, the display 214 can be a touchscreen, where the user interface 218 is displayed on the touchscreen.

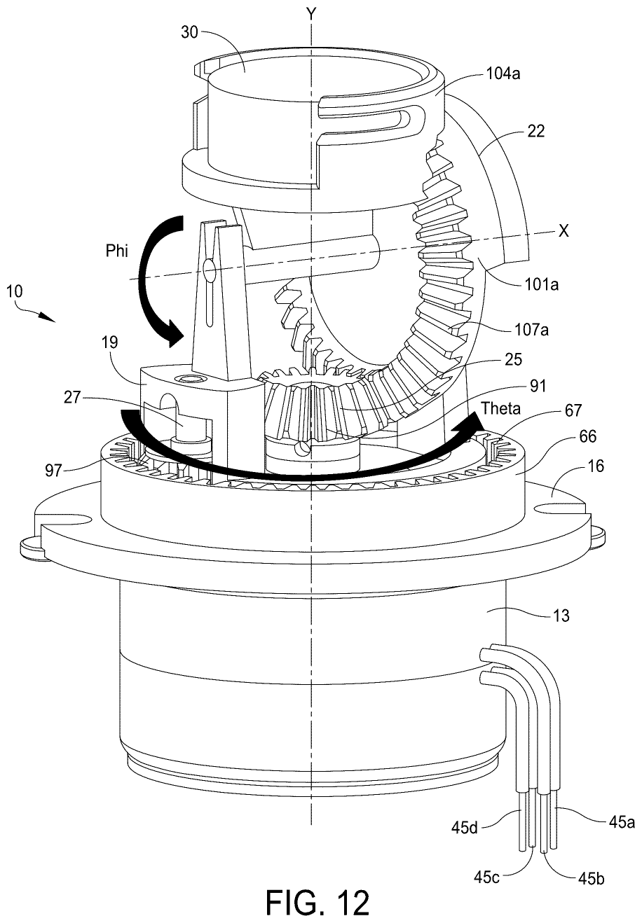

FIG. 12 illustrates an isometric view of the scanner system 10 in accordance with some embodiments. As discussed above, the scanner system 10 may be a part of a medical device, such as a bladder scanner. In some embodiments, the scanner system 10 may be configured to scan a portion of a patient's anatomy (e.g., a patient's bladder when the scanner system 10 is integrated with a bladder scanner) according to a predetermined sequence. The scanner system 10 may perform the predetermined sequence in response to a command from a button on the medical device, a command from a computing device (e.g., computing device 200) communicatively coupled to the scanner system 10, or a robotic system communicatively coupled to the scanner system 10, or any combination thereof.

The predetermined sequence may include one or more rotations of the sensor 30 about a first axis (e.g., the X-axis) in the phi (φ) direction as illustrated in FIG. 12. The predetermined sequence may include one or more rotations of the sensor 30 about a second axis (e.g., the Y-axis) in the theta (θ) direction as illustrated in FIG. 12. In some embodiments, the predetermined sequence may include rotating the shaft 36 in a first direction and rotating the shaft 36 in a second direction. In some embodiments, the predetermined sequence may be 1-3 seconds, 0.5-3.5 seconds, or 0.2-5 seconds. In some embodiments, the predetermined sequence may be about 1.6 seconds. It will be appreciated that the predetermined sequence may be less than 0.2 seconds or more than 5 seconds in some embodiments.

FIG. 13 illustrates a side view of the scanner system 10 in accordance with some embodiments. As discussed above, the motor 13 of the scanner system 10 may be configured to apply a plurality of torques to the shaft 36. In response to a first torque applied, the shaft 36 drives the bevel gear 101a through the meshing of the plurality of teeth 91 of the pinion gear 25 and the plurality of teeth 107a of the bevel gear 101a. At a predetermined angle of the sensor 30, the stop 109 engages the first post 71a, preventing further movement of the assembly 22. At this point, the motor 13 applies a second torque value to the shaft 36. For example, for a current controlled motor, the torque value may change based on a change in current. As another example, for a voltage controlled motor, the torque value may change based on the voltage applied. As yet another example, a predetermined table may be used to run the controller or computing device to control the torque values applied. In some embodiments, the control of the motor 13 torque values may be adaptive, facilitated by one or more sensors configured to sense various parameters of the motor 13 (e.g., voltage, current, resistance, RPM, torque, etc.).

With the assembly 22 fixed against the stand 19, the second torque value applied to the shaft 36 overcomes the friction force of the friction member 99. With the friction force overcome, the stand 19 is configured to move, facilitated by the meshing of the plurality of teeth 97 of the planetary gear 27 and the plurality of teeth 67 of the ring gear 66. In some embodiments, the second torque value is higher than the first torque value. The scanner system 10 may be configured to alternate between applying the first torque value to rotate the sensor 30 in the φ direction and applying the second torque value to rotate the sensor 30 in the θ direction according to the predetermined sequence. In some embodiments, the scanner system 10 is configured to alternate direction of the shaft 36 to change the scanning direction in both of the φ and θ rotational directions (i.e., about either side of the X-axis and Y-axis). In some embodiments, the number of scans may be given by:

$$\# of scan lines / plane \qquad ( Equation 1 )$$

As an example, the number of scan lines may be 70-90 scan lines, 50-100 scan lines, 25-125 scan lines, or 10-150 scan lines. It will be appreciated that the number of scan lines may be less than 10 or more than 150 in some embodiments. In some embodiments, the number of scan lines may be about 80 scan lines. The number of planes of the scan may depend on the direction of rotation. For example, the phi (φ) direction or the theta (θ) direction may define 1-360 planes. In some embodiments, with a fixed theta position, the phi direction may define just a single plane. With a fixed phi position, the theta (θ) direction may define one or more planes (e.g., 1-360 planes, 2-180 planes, 6-60 planes, etc.). In some embodiments, the theta direction may define 12 planes. It will be appreciated that the phi or the theta direction may define more than 360 planes.

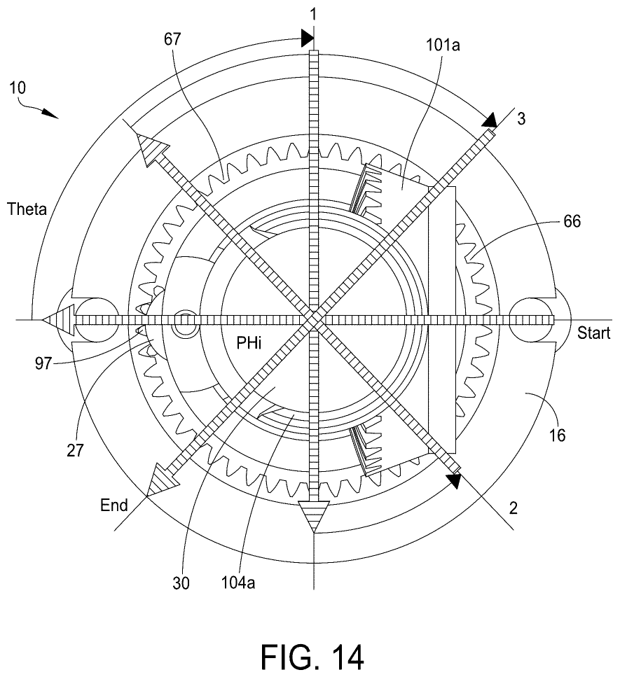

FIG. 14 illustrates an exemplary predetermined sequence of the scanner system 10 in accordance with some embodiments. As illustrated in FIG. 14, the predetermined sequence may start with applying the first torque value to the shaft 36 in a first direction to rotate the assembly 22 in a first φ rotational direction. Rotation of the assembly 22 in the φ direction may include rotating the sensor 30 between 90-150 degrees in the first φ rotational direction until the stop 109 engages the stand 19. The motor 13 may then switch to applying the second torque value to the shaft 36 in the first direction, rotating the stand 19 in a first θ rotational direction to position (1). The rotation of the stand 19 may be between 15-90 degrees in the first θ rotational direction. In some embodiments, the amount of rotation of the stand 19 may depend on the number of planes the sensor 30 is supposed to scan. For example, the amount of rotation of the stand 19 may depend on the following equation:

$$\frac{180 °}{\# of planes} \qquad ( Equation 2 )$$

Once the stand 19 reaches position (1), the motor 13 may switch back to applying the first torque value to the shaft 36 in a second direction. Rotation of shaft 36 at the first torque value in the second direction rotates the assembly 22 in a second φ rotational direction. Rotation of the assembly 22 in the second φ rotational direction may include rotating the sensor 30 between 90-150 degrees in the second φ rotational direction until the stop 109 engages the stand 19. The motor 13 may then switch to applying the second torque value to the shaft 36 in the second direction, rotating the stand 19 in a second θ rotational direction to position (2). The rotation of the stand 19 may be between 15-90 degrees in the second θ rotational direction.

Once the stand 19 reaches position (2), the motor 13 may switch back to applying the first torque value to the shaft 36 in the first direction again. Rotation of shaft 36 at the first torque value in the first direction rotates the assembly 22 in the first φ rotational direction. Rotation of the assembly 22 in the first φ rotational direction may include rotating the sensor 30 between 90-150 degrees in the first φ rotational direction until the stop 109 engages the stand 19. The motor 13 may then switch to applying the second torque value to the shaft 36 in the first direction, rotating the stand 19 in the first θ rotational direction to position (3). The rotation of the stand 19 may be between 15-90 degrees in the first θ rotational direction.

Once the stand 19 reaches position (3), the motor 13 may switch back to applying the first torque value to the shaft 36 in the second direction. Rotation of shaft 36 at the first torque value in the second direction rotates the assembly 22 in the second φ rotational direction. Rotation of the assembly 22 in the second φ rotational direction may include rotating the sensor 30 between 90-150 degrees in the second φ rotational direction until the stop 109 engages the stand 19. In some embodiments, the predetermined sequence ends. In some embodiments, the predetermined sequence described above will restart from the beginning. However, it will be appreciated that the predetermined sequence may continue until all scans of the patient's anatomy are complete. The predetermined sequence described above regarding FIG. 14 is merely an example. Other sequences or modifications to the predetermined sequence may be employed.

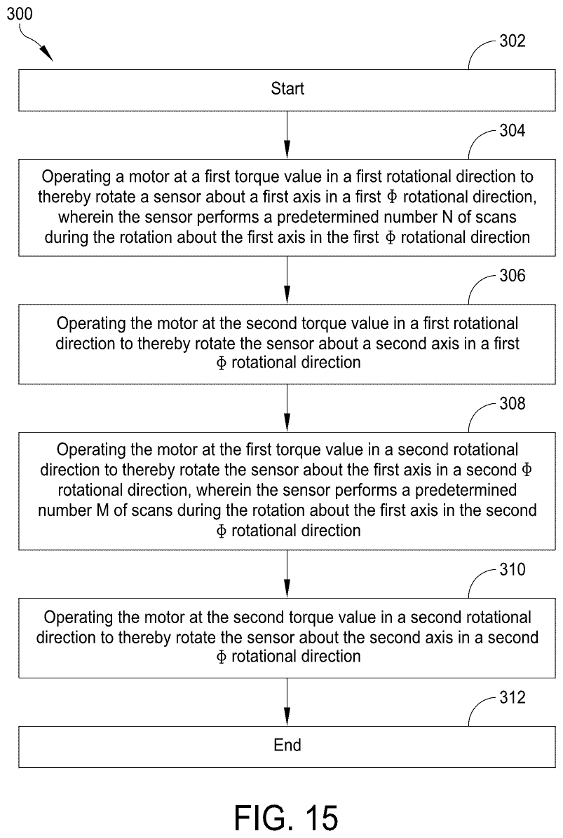

FIG. 15 illustrates a first exemplary method 300 of performing a plurality of scans in a three-dimensional space in accordance with some embodiments. The method 300 may start with step 302. The method 300 may also comprise step 304, which may include operating a motor 13 at a first torque value in a first rotational direction to thereby rotate a sensor 30 about a first axis in a first φ rotational direction. The sensor 30 may perform a predetermined number N of scans during the rotation about the first axis in the first φ rotational direction. The method 300 may also comprise step 306, which may include operating the motor 13 at a second torque value in the first rotational direction to thereby rotate the sensor 30 about a second axis in a first θ rotational direction. The method 300 may also comprise step 308, which may include operating the motor 13 at the first torque value in a second rotational direction to thereby rotate the sensor 30 about the first axis in a second φ rotational direction. The sensor 30 may perform a predetermined number M of scans during the rotation about the first axis in the second φ rotational direction. The method 300 may also comprise step 310, which may include operating the motor 13 at the second torque value in the second rotational direction to thereby rotate the sensor 30 about the second axis in a second θ rotational direction. The method 300 may end at step 312.

In some embodiments, the amount of rotation of the sensor 30 in the second θ rotational direction may be less than the amount of rotation of the sensor 30 in the first θ rotational direction. In some embodiments, the amount of rotation of the sensor 30 in the second θ rotational direction may be equal to half of the amount of rotation of the sensor 30 in the first θ rotational direction. In some embodiments, the amount of rotation of the sensor 30 in the first θ rotational direction may be approximately 60 degrees and the amount of rotation of the sensor 30 in the second θ rotational direction may be approximately 30 degrees. In some embodiments, the amount of rotation of the sensor 30 in the first θ rotational direction may be approximately 30 degrees and the amount of rotation of the sensor 30 in the second θ rotational direction may be approximately 15 degrees. In some embodiments, the first axis may be orthogonal to the second axis. In some embodiments, the rotation of the sensor 30 about the first axis may be independent of the rotation of the sensor 30 about the second axis.

In some embodiments, the motor 13 may apply the first torque value at time t₁ to rotate the sensor 30 about the first axis, and the motor 13 may apply the second torque value at time t₂ to rotate the sensor 30 about the second axis. In some embodiments, the second torque value may be greater than the first torque value. In some embodiments, N is 10-150 scans. In some embodiments, N is 25-125 scans. In some embodiments, N is 50-100 scans. In some embodiments, N is 70-90 scans. In some embodiments, N is approximately 80 scans. It will be appreciated that the number of N scans may be less than 10 or more than 150 scans. In some embodiments, N≠M.

In some embodiments, the sensor 30 may be configured to rotate at least 120 degrees about the second axis. In some embodiments, the sensor 30 may be configured to rotate at least 90 degrees about the second axis. In some embodiments, the sensor 30 may be configured to rotate approximately 150 degrees about the first axis and the sensor 30 may be configured to rotate approximately 135 degrees about the second axis.

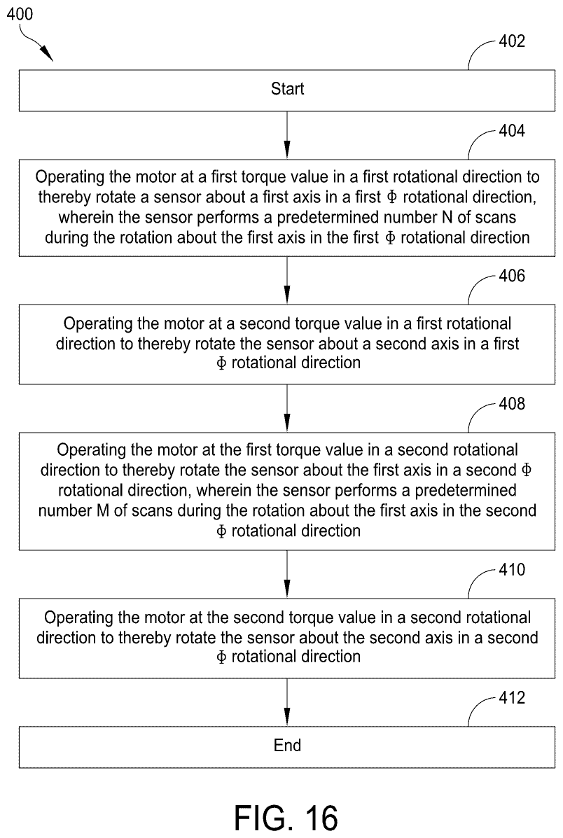

FIG. 16 illustrates a second exemplary method 400 of performing a plurality of scans in a three-dimensional space in accordance with some embodiments. In some embodiments, a method 400 of performing a plurality of scans in a three-dimensional space may use a single motor scanner system 10 having a motor 13 shaft 36, a body 16 having a first gear 66 having a first plurality of teeth 67, a stand 19, and an assembly 22 rotatably coupled to the stand 19. The assembly 22 may have a sensor 30 and a second gear 101a having a second plurality of teeth 107a. The scanner system 10 may also have a third gear 25 having a third plurality of teeth 91. The third gear 25 may be coupled to the motor 13 shaft 36. The third plurality of teeth 91 may mesh with the second plurality of teeth 107a. The scanner system 10 may also include a fourth gear 27 having a fourth plurality of teeth 97. The fourth plurality of teeth 97 may mesh with the first plurality of teeth 67. The scanner system 10 may also include a friction member 99 biased between the stand 19 and the fourth gear 27.

The method 400 may start at step 402. The method 400 may also comprise step 404, which may include operating the motor 13 at a first torque value in a first rotational direction to thereby rotate the sensor 30 about a first axis in a first φ rotational direction. The sensor 30 may perform a predetermined number N of scans during the rotation about the first axis in the first φ rotational direction. The method 400 may also comprise step 406, which may include operating the motor 13 at a second torque value in the first rotational direction to thereby rotate the sensor 30 about a second axis in a first θ rotational direction. The method 400 may also comprise step 408, which may include operating the motor 13 at the first torque value in a second rotational direction to thereby rotate the sensor 30 about the first axis in a second φ rotational direction. The sensor 30 may perform a predetermined number M of scans during the rotation about the first axis in the second φ rotational direction. The method 400 may also comprise step 410, which may include operating the motor 13 at the second torque value in the second rotational direction to thereby rotate the sensor 30 about the second axis in a second θ rotational direction. The method 400 may end at step 412.

In some embodiments, the amount of rotation of the sensor 30 in the second θ rotational direction may be less than the amount of rotation of the sensor 30 in the first θ rotational direction. In some embodiments, the amount of rotation of the sensor 30 in the second θ rotational direction may be equal to half of the amount of rotation of the sensor 30 in the first θ rotational direction. In some embodiments, the amount of rotation of the sensor 30 in the first θ rotational direction may be approximately 60 degrees and the amount of rotation of the sensor 30 in the second θ rotational direction may be approximately 30 degrees. In some embodiments, the amount of rotation of the sensor 30 in the first θ rotational direction may be approximately 30 degrees and the amount of rotation of the sensor 30 in the second θ rotational direction may be approximately 15 degrees. In some embodiments, the first axis may be orthogonal to the second axis. In some embodiments, the rotation of the sensor 30 about the first axis may be independent of the rotation of the sensor 30 about the second axis.

In some embodiments, the motor 13 may apply the first torque value at time t₁ to rotate the sensor 30 about the first axis, and the motor 13 may apply the second torque value at time t₂ to rotate the sensor 30 about the second axis. In some embodiments, the second torque value may be greater than the first torque value. In some embodiments, N is 10-150 scans. In some embodiments, N is 25-125 scans. In some embodiments, N is 50-100 scans. In some embodiments, N is 70-90 scans. In some embodiments, N is approximately 80 scans. It will be appreciated that the number of N scans may be less than 10 or more than 150 scans. In some embodiments, N≠M.

In some embodiments, the sensor 30 may be configured to rotate at least 120 degrees about the second axis. In some embodiments, the sensor 30 may be configured to rotate at least 90 degrees about the second axis. In some embodiments, the sensor 30 is configured to rotate approximately 150 degrees about the first axis and the sensor 30 is configured to rotate approximately 135 degrees about the second axis.

In addition, the methods and system described herein can be at least partially embodied in the form of computer-implemented processes and apparatus for practicing those processes. The disclosed methods may also be at least partially embodied in the form of tangible, non-transitory machine-readable storage media encoded with computer program code. For example, the steps of the methods can be embodied in hardware, in executable instructions executed by a processor (e.g., software), or a combination of the two. The media may include, for example, RAMs, ROMs, CD-ROMs, DVD-ROMs, BD-ROMs, hard disk drives, flash memories, or any other non-transitory machine-readable storage medium. When the computer program code is loaded into and executed by a computer, the computer becomes an apparatus for practicing the method. The methods may also be at least partially embodied in the form of a computer into which computer program code is loaded or executed, such that, the computer becomes a special purpose computer for practicing the methods. When implemented on a general-purpose processor, the computer program code segments configure the processor to create specific logic circuits. The methods may alternatively be at least partially embodied in application specific integrated circuits for performing the methods.

It may be emphasized that the above-described embodiments, particularly any “preferred” embodiments, are merely possible examples of implementations, set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

While this specification contains many specifics, these should not be construed as limitations on the scope of any disclosures, but rather as descriptions of features that may be specific to a particular embodiment. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

1. A system, comprising:
a motor comprising a shaft;
a body comprising a first gear having a first plurality of teeth;
a stand;
an assembly rotatably coupled to the stand, the assembly comprising a second gear having a second plurality of teeth;
a third gear having a third plurality of teeth, the third gear coupled to the shaft and the third plurality of teeth meshing with the second plurality of teeth;
a fourth gear having a fourth plurality of teeth, the fourth plurality of teeth meshing with the first plurality of teeth; and
a friction member biased between the stand and the fourth gear.

2. The system of claim 1, wherein the motor is a stepper motor.

3. The system of claim 1, wherein the motor is configured to apply a plurality of torque values to the third gear via the shaft.

4. The system of claim 3, wherein the motor is configured to apply the plurality of torque values in accordance with a predetermined sequence.

5. The system of claim 4, wherein the predetermined sequence includes the motor rotating the shaft in a first direction and rotating the shaft in a second direction.

6. The system of claim 4, wherein the predetermined sequence is less than 2 seconds in duration.

7. The system of claim 1, wherein the motor applies a first torque via the shaft to the third gear thereby rotating the assembly about a first axis.

8. The system of claim 7, wherein the friction member prevents the first torque from rotating the body about a second axis.

9. The system of claim 8, wherein the motor applies a second torque via the shaft to the third gear, wherein the second torque is greater than the first torque, thereby rotating the body about the second axis.

10. The system of claim 9, wherein the assembly comprises a structure configured to interact with the stand after the assembly rotates a predetermined amount about the first axis.

11. The system of claim 8, wherein the first axis is orthogonal to the second axis.

12. (canceled)

13. The system of claim 1, wherein the assembly is configured to rotate at least 90 degrees about the first axis.

14. (canceled)

15. The system of claim 1, wherein the body is configured to rotate at least 90 degrees about the second axis.

16. The system of claim 1, wherein the assembly is configured to rotate approximately 150 degrees about the first axis and the body is configured to rotate approximately 135 degrees about the second axis.

17. The system of claim 1, wherein the friction member is one of a spring or a ratchet and pawl mechanism.

18. (canceled)

19. The system of claim 1, wherein the first gear is a ring gear, the second gear is a bevel gear, the third gear is a pinion gear, and the fourth gear is a planetary gear.

20. The system of claim 1, wherein the assembly is configured to hold a sensor.

21. The system of claim 19, wherein the sensor is an ultrasound probe.

22-26. (canceled)

27. A scanner system comprising:
a motor comprising a shaft;
a body comprising a first gear having a first plurality of teeth;
a stand;
an assembly rotatably coupled to the stand, the assembly comprising a sensor and a second gear having a second plurality of teeth;
a third gear having a third plurality of teeth, the third gear coupled to the shaft and the third plurality of teeth meshing with the second plurality of teeth;
a fourth gear having a fourth plurality of teeth, the fourth plurality of teeth meshing with the first plurality of teeth; and
a friction member biased between the stand and the fourth gear,
wherein the motor is a stepper motor and is configured to apply a plurality of torque values to the third gear via the shaft.

28-50. (canceled)

51. A method of performing a plurality of scans in a three-dimensional space, the method comprising the steps of:
operating a motor at a first torque value in a first rotational direction to thereby rotate a sensor about a first axis in a first φ rotational direction, wherein the sensor performs a predetermined number N of scans during the rotation about the first axis in the first φ rotational direction;
operating the motor at a second torque value in the first rotational direction to thereby rotate the sensor about a second axis in a first θ rotational direction;
operating the motor at the first torque value in a second rotational direction to thereby rotate the sensor about the first axis in a second φ0 rotational direction, wherein the sensor performs a predetermined number M of scans during the rotation about the first axis in the second φ rotational direction; and
operating the motor at the second torque value in the second rotational direction to thereby rotate the sensor about the second axis in a second θ rotational direction.

52-80. (canceled)
